(19) United States
(12) Patent Application Publication
(10) Pub. No.: US 2017/0333648 A1
(43) Pub. Date: Nov. 23, 2017

(54) AMPOULE FOR INHALANT DEVICE

(71) Applicants: KATHRYN CASHMAN, NASHVILLE, TN (US); MICHAEL HAARLANDER, NASHVILLE, TN (US)

(72) Inventors: KATHRYN CASHMAN, NASHVILLE, TN (US); MICHAEL HAARLANDER, NASHVILLE, TN (US)

(21) Appl. No.: 15/673,276

(22) Filed: Aug. 9, 2017

Related U.S. Application Data
(63) Continuation-in-part of application No. 15/587,151, filed on May 4, 2017.
Application No. 15/587,151 is a continuation-in-part of application No. 15/215,718, filed on Jul. 21, 2016.
Application No. 15/215,718 is a continuation-in-part of application No. 15/184,761, filed on Jun. 16, 2016.
(60) Provisional application No. 62/372,374, filed on Aug. 9, 2016.
(60) Provisional application No. 62/302,484, filed on Mar. 2, 2016.
(60) Provisional application No. 62/180,591, filed on Jun. 16, 2015.
(60) Provisional application No. 62/194,814, filed on Jul. 21, 2015.
(60) Provisional application No. 62/331,766, filed on May 4, 2016.

(51) Int. Cl. A61M 15/00; B05B 17/00; A61K 9/00; A61M 11/00

(52) U.S. Cl. CPC A61M 15/0085; A61K 9/0078; B05B 17/0646; A61K 9/0043; A61M 11/005

(57) ABSTRACT

An ampoule with an integrated piezoelectric unit for use with a personal nebulizer or vaporizing unit with a mouthpiece. The substance to be vaporized is contained in the ampoule which is inserted into the unit, where the substance is vaporized through the integrated piezoelectric transducers or atomizers providing sonic or ultrasonic vibration. The substance to be vaporized may include a variety of therapeutic, homeopathic, or naturopathic formulations, remedies, or serums.

This application claims benefit of and priority to U.S. Provisional Application No. 62/372,374, filed Aug. 9, 2016, and is a continuation-in-part application of U.S. patent application Ser. No. 15/587,151, filed May 4, 2017, which claims benefit to U.S. Provisional Application No. 62/331,766, filed May 4, 2016, and which also is a continuation-in-part application of U.S. patent application Ser. No. 15/215,718, filed Jul. 21, 2016, which is a continuation-in-part application of U.S. patent application Ser. No. 15/184,761, filed Jun. 16, 2016, which claim benefit of and priority to U.S. Provisional Applications Nos. 62/302,484, filed Mar. 2, 2016, and 62/180,591, filed Jun. 16, 2015, and U.S. Provisional Application No. 62/194,814, filed Jul. 21, 2015, and is entitled to those filing dates for priority. The specifications, figures, appendices and complete disclosures of U.S. patent application Ser. Nos. 15/184,761 and 15/215,718 and 15/587,151, and U.S. Provisional Applications Nos. 62/372,374; 62/331,766; 62/320,484; 62/194,814; and 62/180,591 are incorporated herein in their entireties by specific reference for all purposes.

### FIELD OF INVENTION

This invention relates to an ampoule for a personal inhalation device. More particular, this invention relates to an ampoule for use with a handheld vaporizing device for generating a vapor for inhaling by a user.

### BACKGROUND OF THE INVENTION

A variety of vaporizers or nebulizers are known in the prior art, particularly for use with tobacco or tobacco extracts. Typically, the substance to be vaporized is heated by the device, and emitted through an opening or orifice. However, such devices do not provide the ability to finely controlled the application of a therapeutic substance, or provide for the calibration and monitoring of treatments using a vaporized therapeutic sub stance.

### SUMMARY OF INVENTION

In various embodiments, the present invention comprises a personal nebulizer or vaporizing unit. While referred to herein as a nebulizer or vaporizing unit, the device encompasses devices of all sorts, including but not limited to inhalers, nebulizers, vaporizers, humidifiers, ventilators, or other devices that are used for delivery of any sort of substance into the body via breathing, and includes but is not limited to devices that break up solutions and suspensions into small aerosol droplets for inhalation by a user.

The unit comprises an outer main shell with a mouthpiece unit at one end. The mouthpiece unit may be detachable and replaceable. In addition, the top of the shell may be removable from the base of the shell. The mouthpiece unit comprises an orifice through which vapor is emitted. A mouthpiece cover may be used to cover some or all of the mouthpiece unit. The cover may snap-fit around the perimeter, or a portion of the cover may be inserted into the orifice, or both, to secure the cover in place.

The outer main shell further includes one or more controls, such as a button, slider, or switch, that may be used to turn the unit on or off, or control other functions, as described below. One or more lights, LEDS, or other indicators may be provided to indicate status of the unit, including, but not limited to, power status and operational status. The main shell further comprises an opening with removable cover for insertion of an ampoule or cartridge into the unit. The cartridge holds the substance to be vaporized during operation of the unit. The vaporized substance is inhaled by the user while holding the unit.

The substance to be vaporized may be in the form of a liquid, gel, gas, solid, or the like. In several embodiments, the substance comprises one or more of a therapeutic substance, homeopathic or naturopathic formulations or remedies, serums, or the like. Particular substances may be chosen or selected for particular desired effects, therapies or treatments, and substances have natural vaporization characteristics that are dependent on a variety of factors, including, but not limited to, temperature, air flow, and substance composition and chemical state. Selection of one or more substances for vaporization may be made based upon information obtained from other devices or systems, such as a breath analysis device that can capture and analyze the content of exhaled air, a health band (e.g., Fit Bit), other detection devices, or information stored in or entered into in a health or wellness computer application, program or database on a computer or mobile device (e.g., smart phone, tablet computer), which can coordinate diagnosing this information and developing recommendations for the substance or substances to be used.

In an alternative embodiment, the unit comprises an internal, refillable chamber for holding the substance to be vaporized. The refillable chamber may be filled by insertion of the substance through the opening with removable cover, or other filling port. In several embodiments, the ampoule itself may comprise a piezoelectric unit, and may be multiple use or single use. The ampoule may be hermetically sealed, and made of any suitable material, including, but not limited to, glass, plastic, polymer, metal, or the like. The ampoule may be rigid, or flexible (e.g., a flexible pouch). In one embodiment, the ampoule comprises a barrel shape with a proximal end and distal end, the proximal end being open and extending into a rim or two opposing tabs perpendicular to the axis of the barrel, each tab comprising an ergonomic and/or flat radius section following the extension point of the tab.

The piezoelectric unit may be located in the top of the ampoule, such as in a cap that screws onto or is otherwise attached to the body of the ampoule. The cap may be permanently affixed to the ampoule, such as by welding, gluing, or adhesive, or may be removable. The piezoelectric unit may be held in an insert or holder used to position the piezoelectric unit directly over the opening of the ampoule, thereby providing consistent flow of the material. The piezoelectric unit may be in the form of a wafer or similar configuration. Power may be supplied to the unit by guide wires or connections to a power source in an inhaler unit in which the ampoule is inserted, or by a battery or similar power source attached to or embedded in the ampoule.

Once the piezoelectric unit is removed, or the cap is opened, the integrity of the electronics is destroyed (e.g., the guide wires are broken or torn) to prevent re-use (i.e., the unit can only be used once with the pre-loaded material, and must be discarded after use). This safeguards against the use of illegal drugs, improper medicaments, or the like. The cap also may be secured.

In additional embodiments, the piezoelectric unit may embedded into the side or bottom of the body of the ampoule in middle or bottom positions. The piezoelectric unit may be inaccessible, ensuring that the ampoule is used only once and cannot be refilled. The exterior of the ampoule may be printed directly onto with a description of the contents, a bar or similar code, and other information.

### BRIEF DESCRIPTION OF THE DRAWINGS

FIG. 1 shows a perspective view of a device in accordance with an embodiment of the present invention.

FIG. 2 shows a top perspective view of the device of FIG. 1.

FIG. 3 shows a top view of the device of FIG. 1.

FIG. 4 shows a side view of the device of FIG. 1.

FIG. 5 shows an exploded perspective view of the device of FIG. 1.

FIG. 6 shows another exploded perspective view of the device of FIG. 1.

FIG. 7 shows a view of the interior of the device of FIG. 1.

FIG. 8 shows another view of the interior of the device of FIG. 1.

FIG. 9 shows a perspective view of the device of FIG. 1 with a cover.

FIG. 10 shows a perspective view of the device of FIG. 1 with light sources.

FIG. 11 shows a perspective view of the device of FIG. 1 with interior light sources.

FIG. 12 shows a view of an ampoule with bar code.

FIG. 13 shows a view of an ampoule with a piezoelectric unit in a top position.

FIG. 14 shows a view of an ampoule with a piezoelectric unit in a middle position.

FIG. 15 shows another view of an ampoule with a piezoelectric unit in a middle position.

FIG. 16 shows a view of an ampoule with a piezoelectric unit in a bottom position.

FIG. 17 shows a view of an ampoule in pouch form with a piezoelectric unit in a top position.

FIG. 18 shows a view of an ampoule in pouch form with a piezoelectric unit in a bottom position.

FIG. 19 shows a view of an ampoule inserted into a personal vaporizing unit.

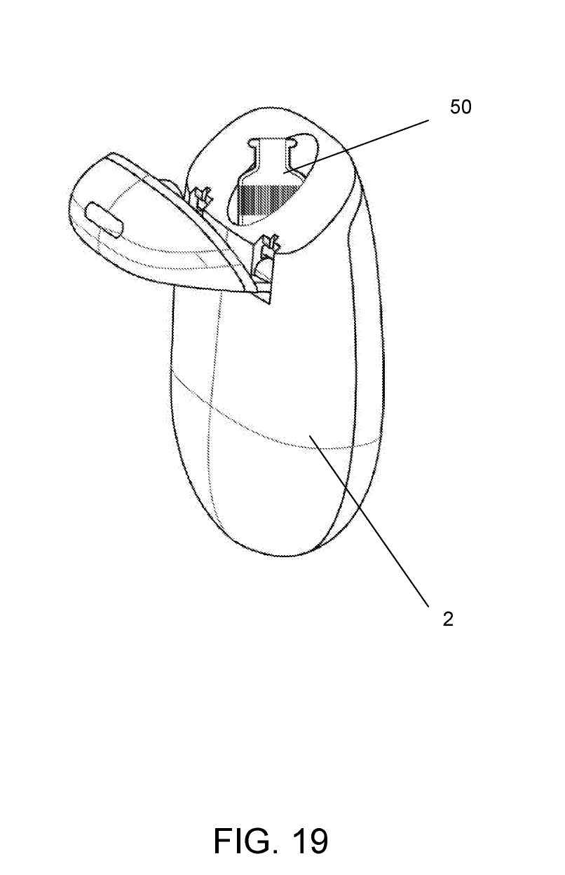

### DETAILED DESCRIPTION OF EXEMPLARY EMBODIMENTS

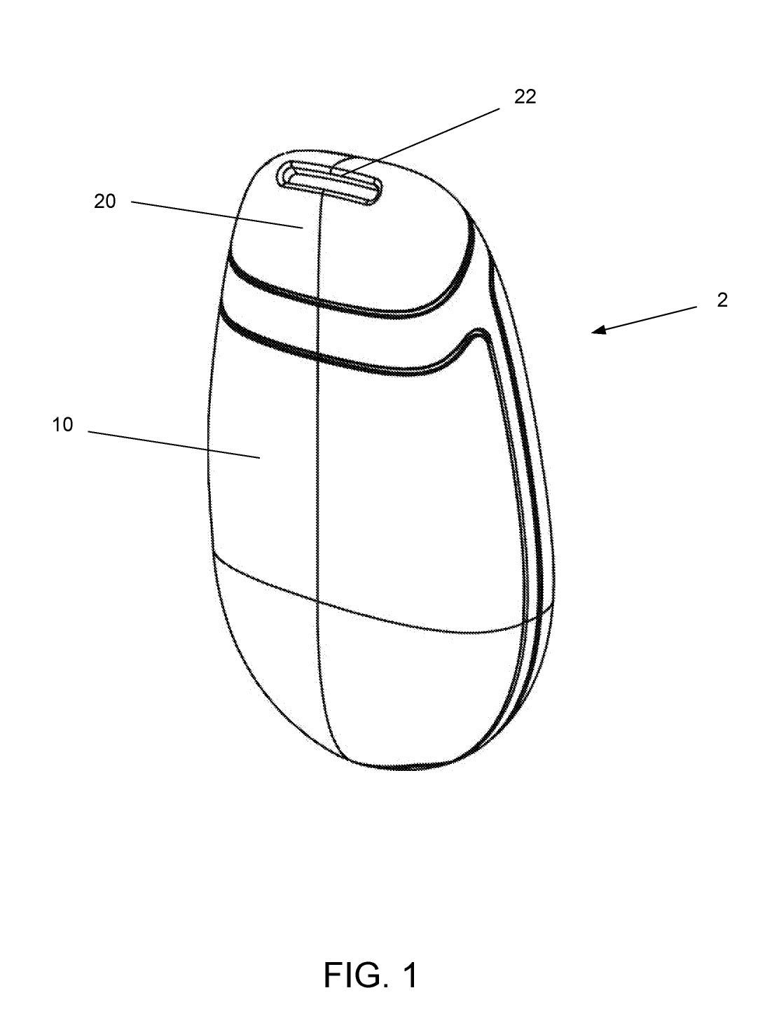

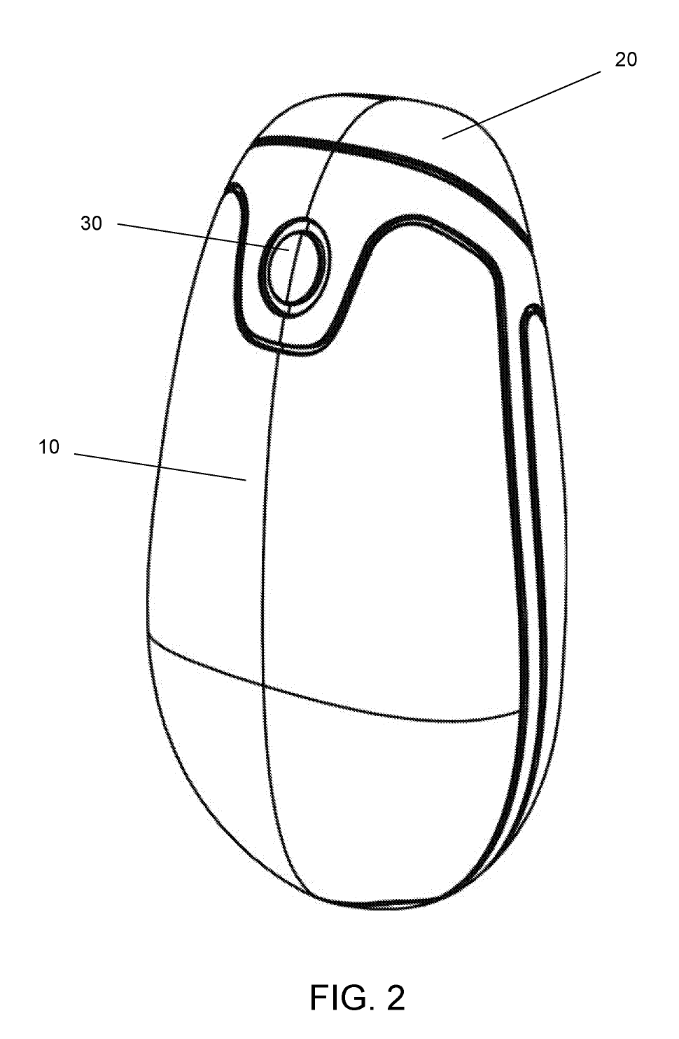

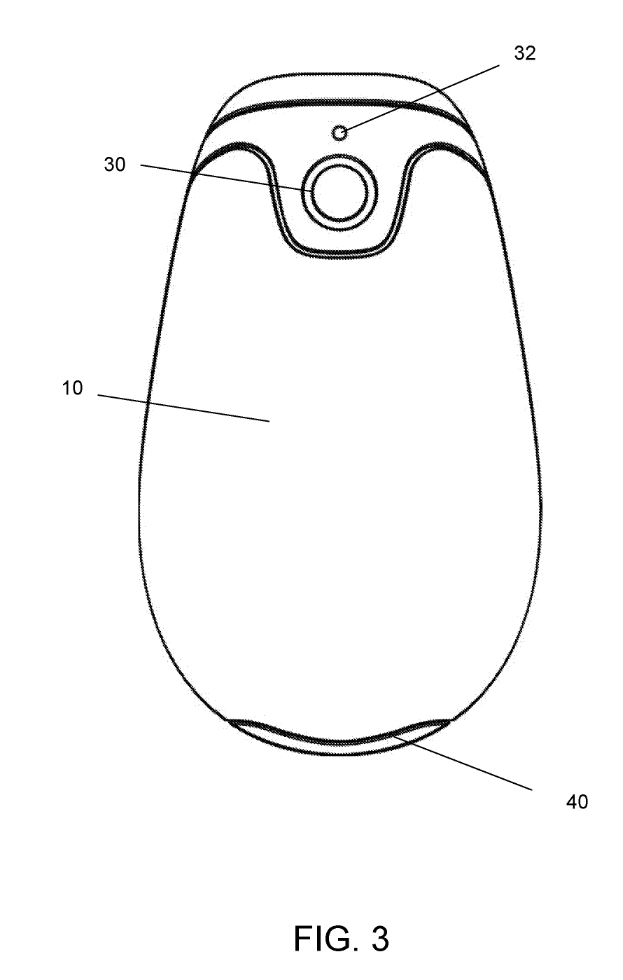

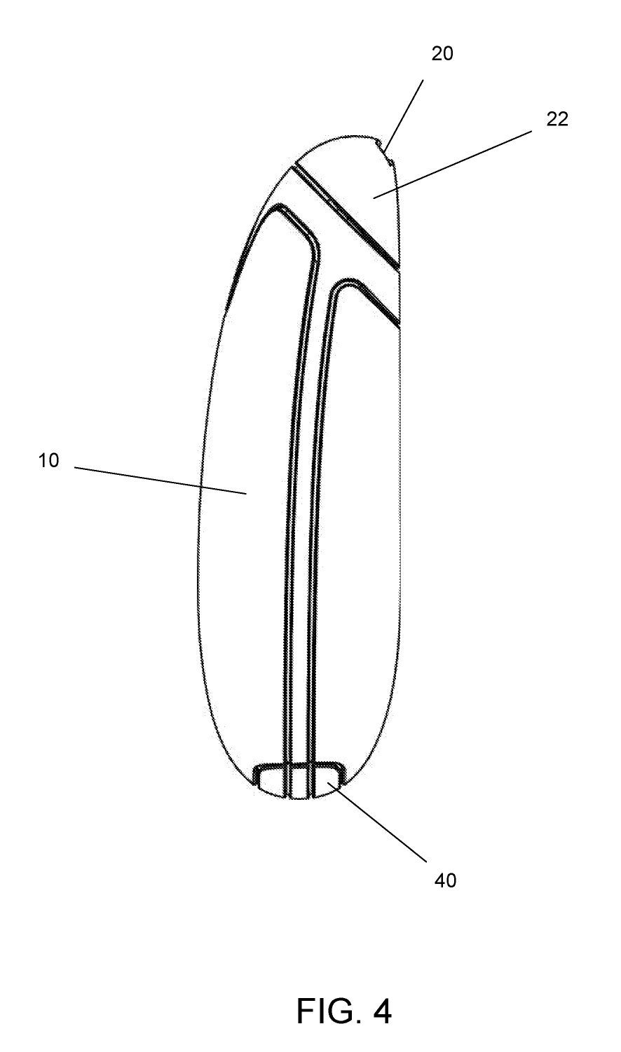

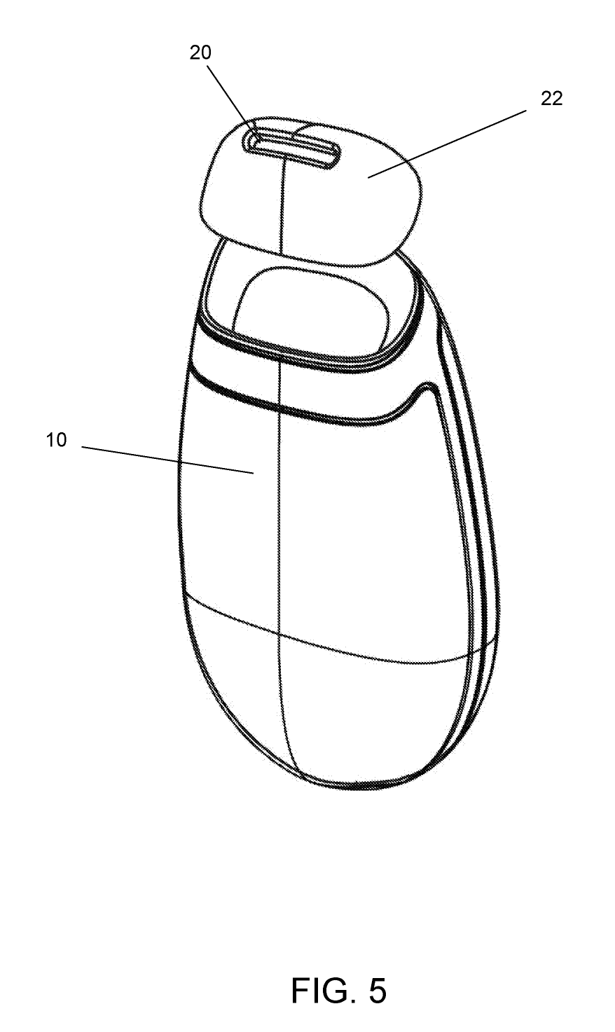

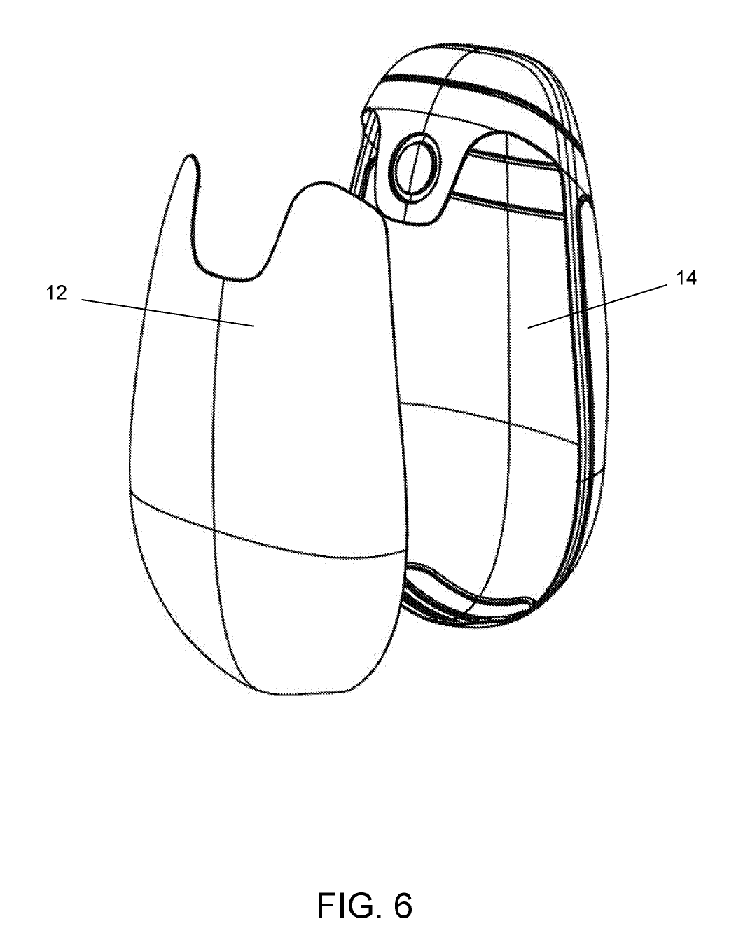

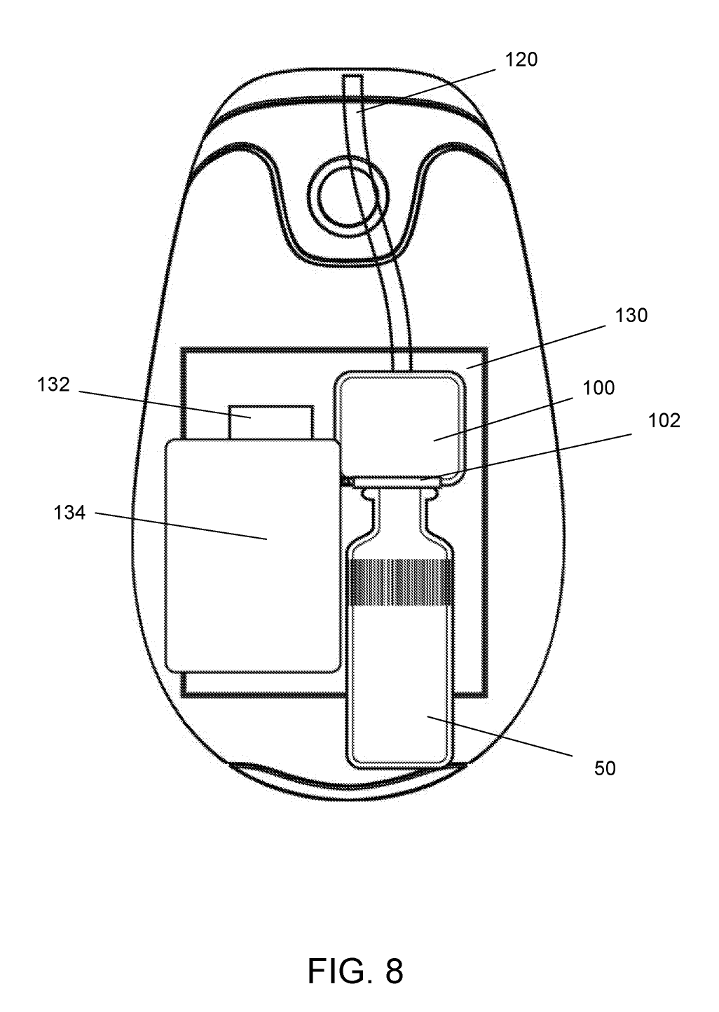

In various exemplary embodiments, as seen in FIGS. 1-8, the present invention comprises an ampoule 50 for use with a personal vaporizing unit 2. The personal vaporizing unit comprises an outer main shell 10 with a mouthpiece unit 20 at one end. The mouthpiece unit may be detachable and replaceable, as seen in FIG. 5. In addition, as in FIG. 6, the top 12 of the shell may be removable from the base 14 of the shell. The top also may be hingedly attached to the base.

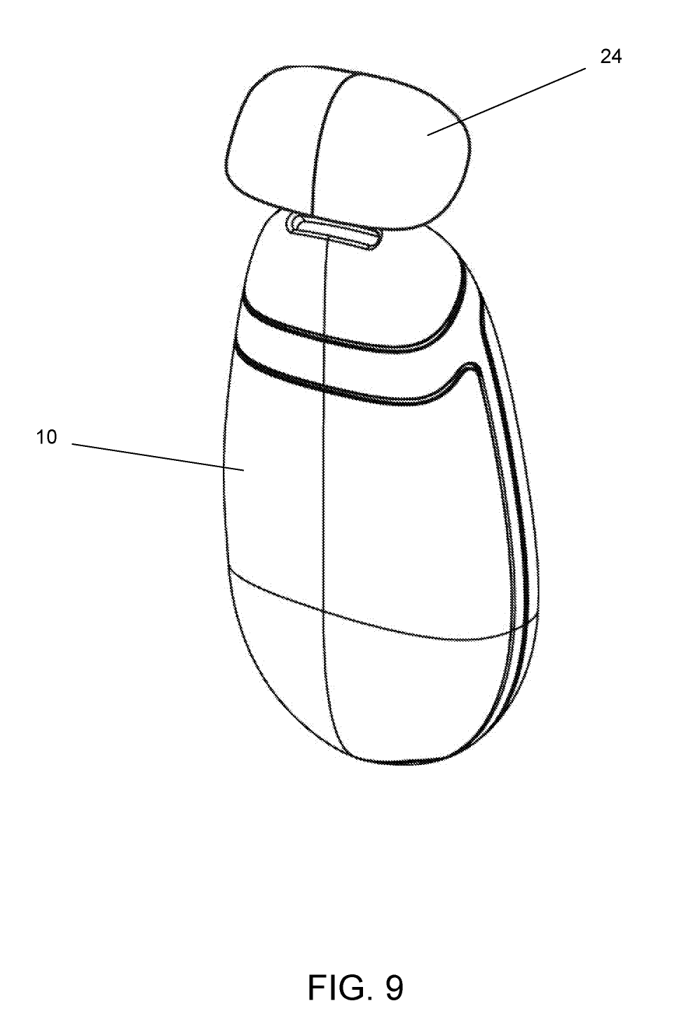

The mouthpiece unit 20 comprise an orifice 22 through which vapor is emitted. A mouthpiece cover 24 may be used to cover some or all of the mouthpiece unit, as seen in FIG. 9. The cover may snap-fit around the perimeter, or a portion of the cover may be inserted into the orifice, or both, to secure the cover in place.

The outer main shell 10 further includes one or more controls 30, such as a button, slider, or switch, that may be used to turn the unit on or off, or control other functions, as described below. One or more lights, LEDS, or other indicators 32 may be provided to indicate status of the unit, including, but not limited to, power status and operational status.

The main shell 10 further comprises an opening with removable cover 40 for insertion of a ampoule 50 into the unit. The ampoule holds the substance to be vaporized during operation of the unit. The vaporized substance is inhaled by the user while holding the unit.

The substance to be vaporized may be in the form of a liquid, gel, gas, solid, or the like. In several embodiments, the substance comprises one or more of a therapeutic substance, homeopathic or naturopathic formulations or remedies, serums, or the like. Particular substances may be chosen or selected for particular desired effects, therapies or treatments, and substances have natural vaporization characteristics that are dependent on a variety of factors, including, but not limited to, temperature, air flow, and substance composition and chemical state. Selection of one or more substances for vaporization may be made based upon information obtained from other devices or systems, such as a breath analysis device that can capture and analyze the content of exhaled air, a health band (e.g., Fit Bit), other detection devices, or information stored in or entered into in a health or wellness computer application, program or database on a computer or mobile device (e.g., smart phone, tablet computer), which can coordinate diagnosing this information and developing recommendations for the substance or substances to be used.

In an alternative embodiment, the personal vaporizing unit comprises an internal, refillable chamber for holding the substance to be vaporized. The refillable chamber may be filled by insertion of the substance through the opening with removable cover 40, or other filling port.

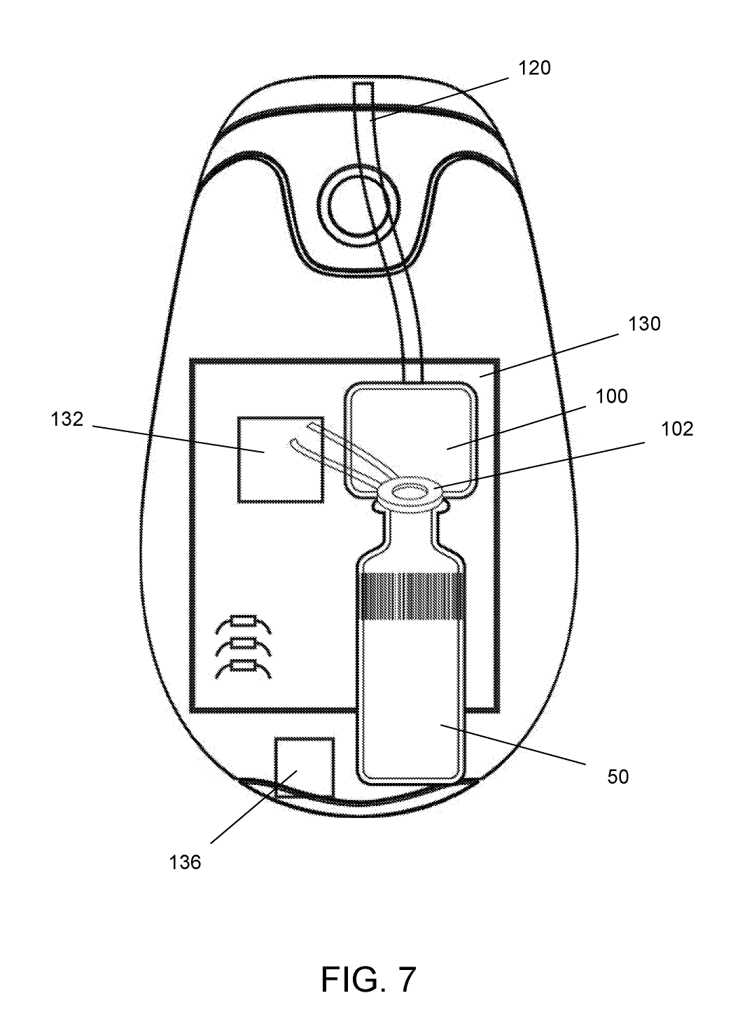

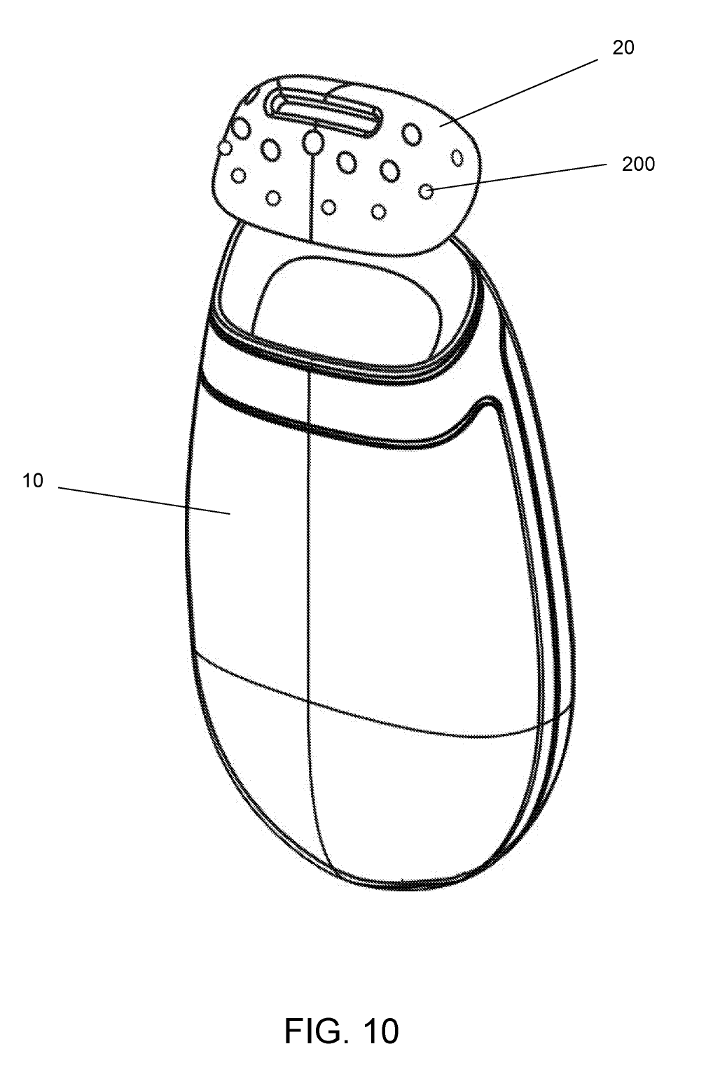

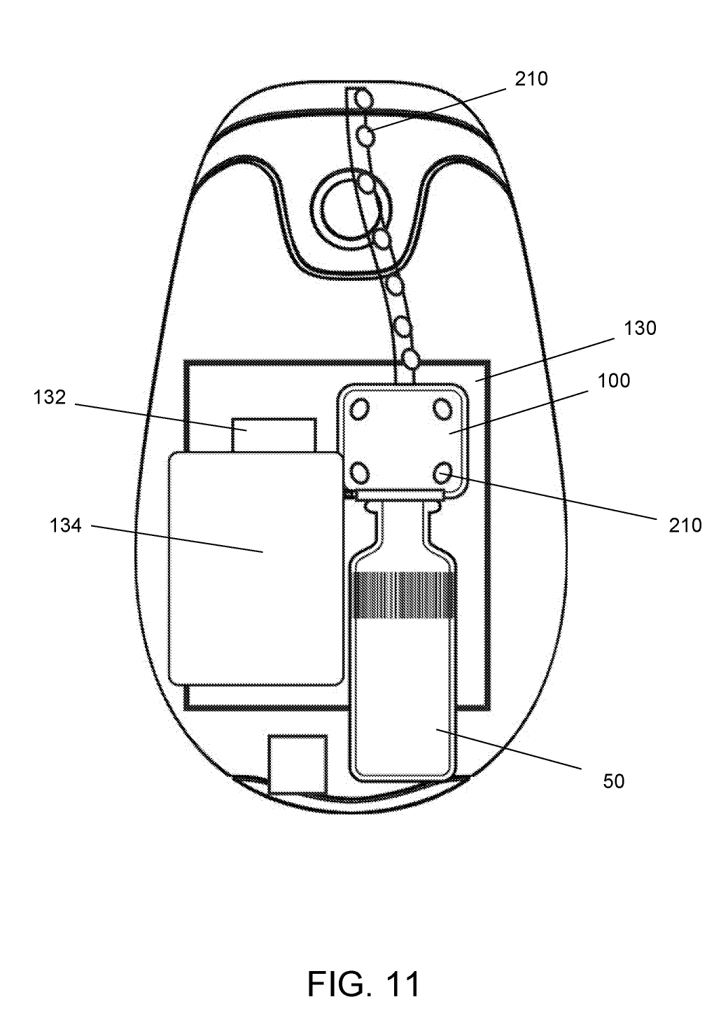

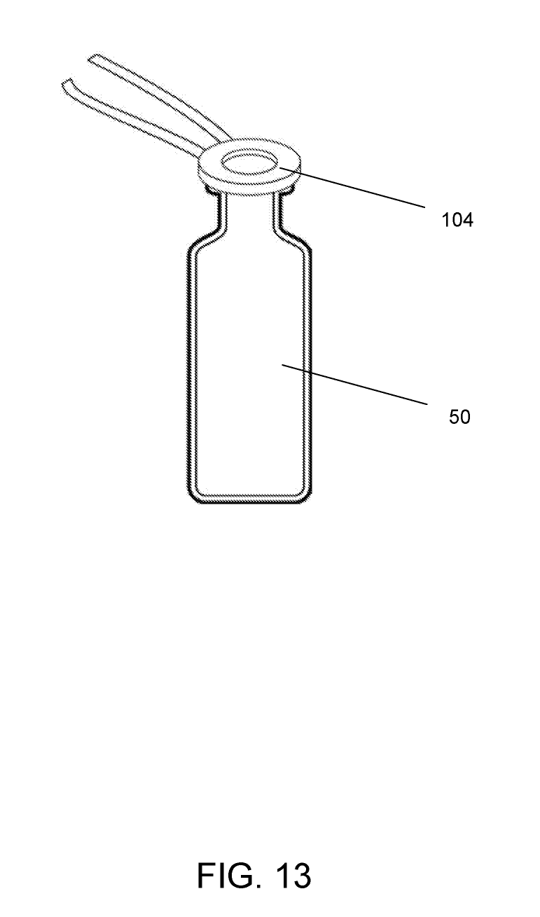

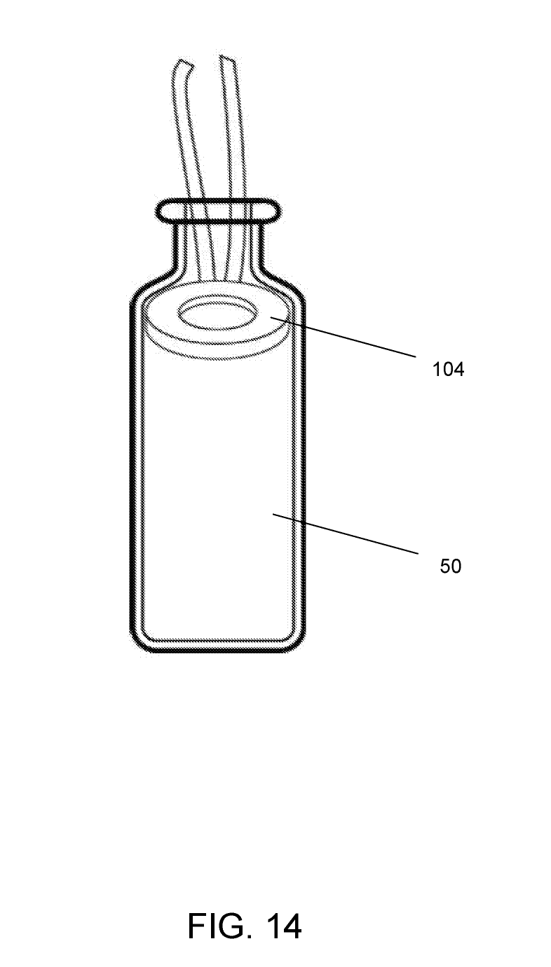

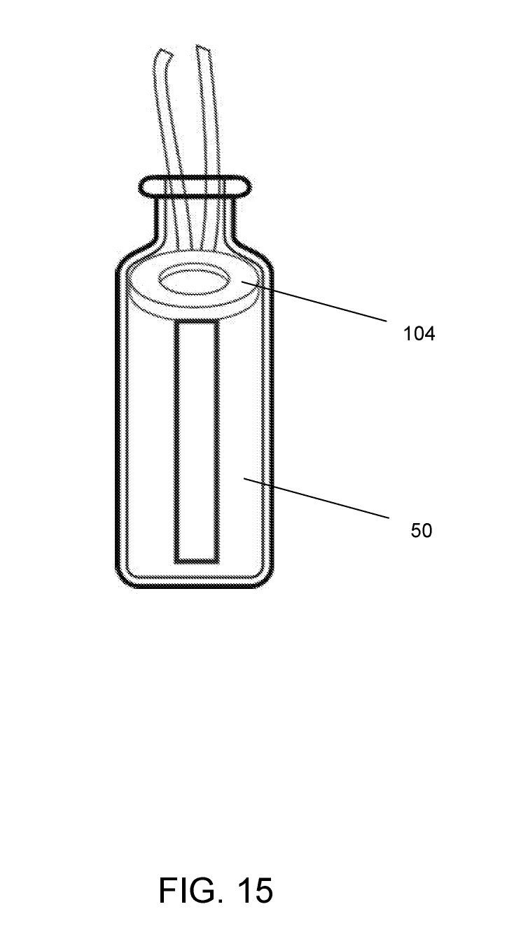

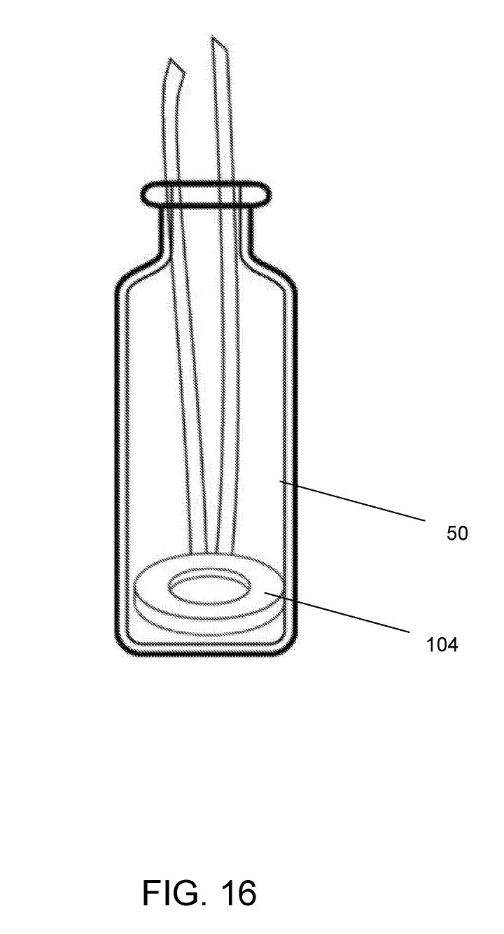

As seen in FIG. 7-18, the interior of the unit comprises a atomization chamber 100, which receives the substance from the ampoule 50 or refillable chamber through such means as a wick or other conduit. Vaporization is achieved by means of one or more piezoelectric transducers or atomizers 102, providing sonic or ultrasonic vibration. Vapor is emitted through conduit 120 to the mouthpiece orifice. Calibration and control of the atomization process (e.g., temperature, size of inlet openings, rate of vaporization, timer) may be controlled by the user manually using a control on the unit, automatically controlled based upon a code or symbol (e.g., bar code) on the ampule, or remotely (such as by wireless connection to a computer or mobile device). This calibration and control may be performed according to the diagnosis and recommendations developed in the manner described above.

A circuit board 130 provides control and power functions. Power may be provided by one or more batteries 134. The battery or batteries may be standard, replaceable batteries, or may be a rechargeable battery built into the unit, and recharged with a recharging cord or similar means. A wireless or Bluetooth chip 132 provides for wireless communications. A USB, mini-USB, or similar communications port 136 provides for direct communications, uploading and downloading of programs or data, and recharging.

Other vaporization means may be provided, such as a heat source (burner, flame, electrical). Temperature may be controlled in the manner of other parameters discussed above.

In yet another embodiment, a unit has multiple vaporization elements, and can vaporize different substances from multiple refillable chambers or multiple ampoules simultaneously, in sequence, or some combination thereof.

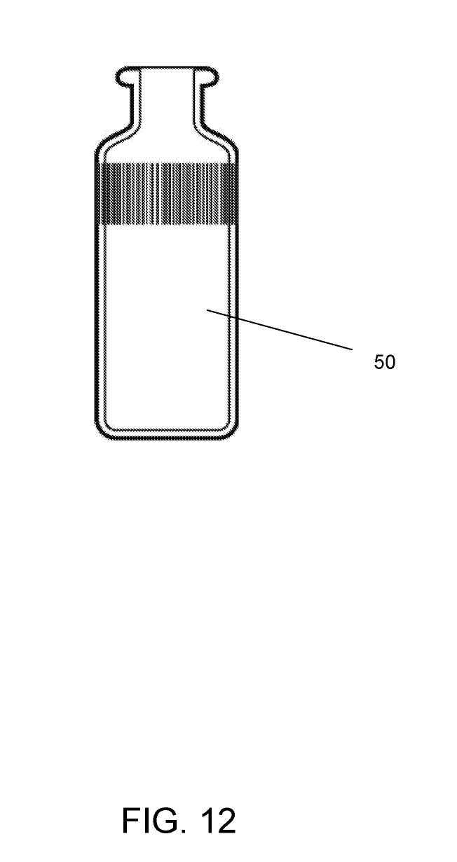

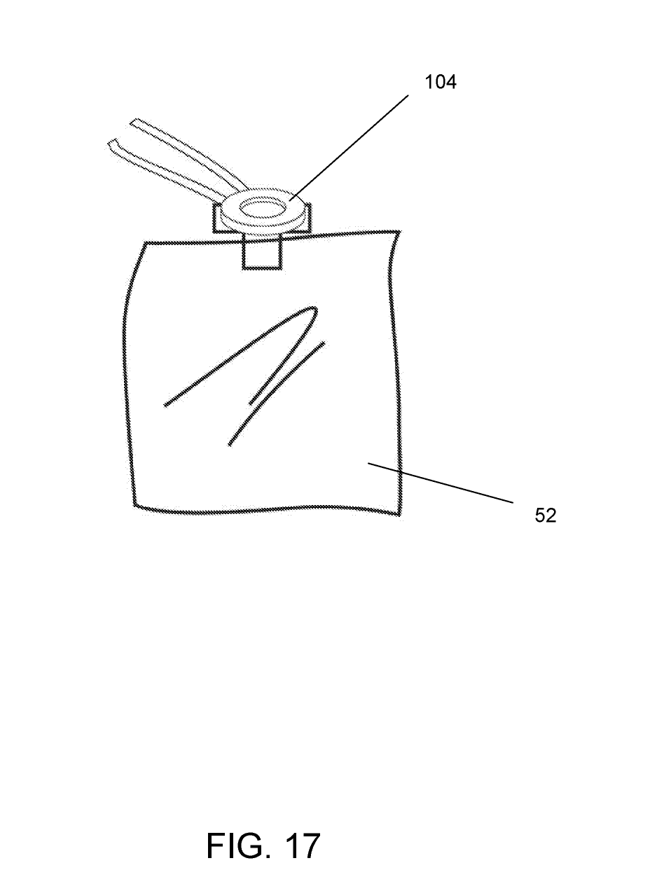

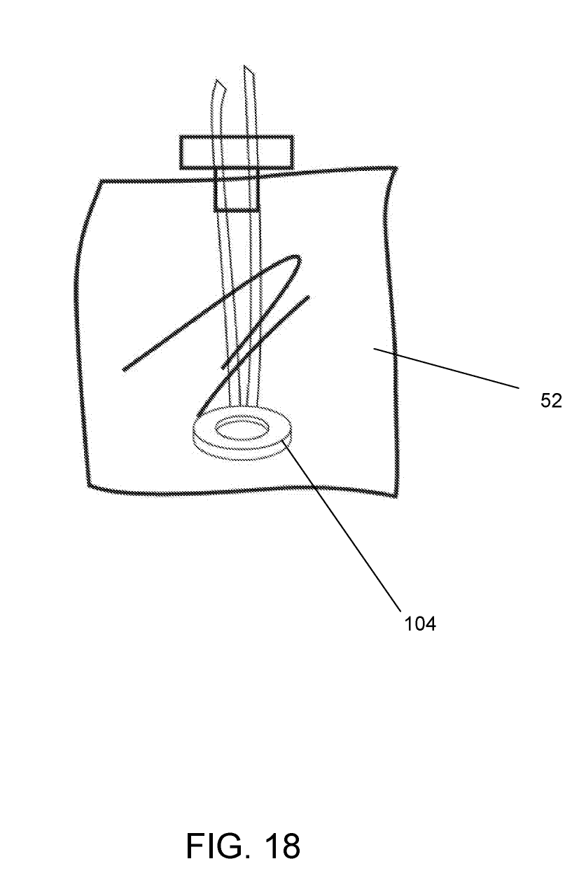

In several embodiments, the ampoule 50 itself may comprise a piezoelectric unit 104, and may be multiple use or single use. The ampoule may be hermetically sealed, and made of any suitable material, including, but not limited to, glass, plastic, polymer, metal, or the like. The ampoule may be rigid, or flexible (e.g., a flexible pouch 52, as seen in FIGS. 17-18). In one embodiment, as seen in FIGS. 12-16 the ampoule comprises a barrel shape with a proximal end and distal end, the proximal end being open and extending into a rim or two opposing tabs perpendicular to the axis of the barrel, each tab comprising an ergonomic and/or flat radius section following the extension point of the tab.

The piezoelectric unit 104 may be located in the top of the ampoule, such as in a cap that screws onto or is otherwise attached to the body of the ampoule. The cap may be permanently affixed to the ampoule, such as by welding, gluing, or adhesive, or may be removable. The piezoelectric unit may be held in an insert or holder used to position the piezoelectric unit directly over the opening of the ampoule, thereby providing consistent flow of the material. The piezoelectric unit may be in the form of a wafer or similar configuration. Power may be supplied to the unit by guide wires or connections to a power source in an inhaler unit in which the ampoule is inserted, or by a battery or similar power source attached to or embedded in the ampoule.

Once the piezoelectric unit is removed, or the cap is opened, the integrity of the electronics is destroyed (e.g., the guide wires are broken or torn) to prevent re-use (i.e., the unit can only be used once with the pre-loaded material, and must be discarded after use). This safeguards against the use of illegal drugs, improper medicaments, or the like.

In additional embodiments, the piezoelectric unit may embedded into the side or bottom of the body of the ampoule in middle or bottom positions. The piezoelectric unit may be inaccessible, ensuring that the ampoule is used only once and cannot be refilled.

The exterior of the ampoule may be printed directly onto with a description of the contents, a bar or similar code, as seen in FIG. 12, and other information.

In yet a further embodiment, the inhalation unit 2 has a computer memory storage capability, and stores vaporization treatment data so that the details of the vaporization treatment can be subsequently used to evaluate clinical or medical treatment compliance and effectiveness. The information may be provided by wired or wireless connection 132, 134 to a health or wellness program, as described above. The unit may have one or more means of wireless communication (e.g., wireless chip, Bluetooth), and wired communication (e.g., data ports, USB ports).

In yet another embodiment, one or more light sources 200 (e.g., LEDs, laser diodes, fiber optics, and the like) may be located on the mouthpiece unit to provide light-related therapy to the mouth, lips and gums. Different arrangements (e.g., colors, intensity, locations) of light sources may be provided on different mouthpiece units, which can be interchanged as desired.

The atomization chamber 100 and conduit 120 may be cleaned between uses using a cleaning solution, by one or more interior UV light or radiation sources 210 along the chamber and conduit, or combinations thereof.

In order to provide a context for the various aspects of the invention, the following discussion provides a brief, general description of a suitable computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, hand-held devices, microprocessor-based systems, multiprocessor systems, TV set-top boxes and devices, programmable consumer electronics, cell phones, personal digital assistants (PDAs), tablets, smart phones, touch screen devices, smart TV, internet enabled appliances, internet enabled security systems, internet enabled gaming systems, internet enabled watches; internet enabled cars (or transportation), network PCs, minicomputers, mainframe computers, embedded systems, virtual systems, distributed computing environments, streaming environments, volatile environments, and the like.

Embodiments of the invention may be implemented in the form of computer-executable instructions, such as program code or program modules, being executed by a computer, virtual computer, or computing device. Program code or modules may include programs, objects, components, data elements and structures, routines, subroutines, functions and the like. These are used to perform or implement particular tasks or functions. Embodiments of the invention also may be implemented in distributed computing environments. In such environments, tasks are performed by remote processing devices linked via a communications network or other data transmission medium, and data and program code or modules may be located in both local and remote computer storage media including memory storage devices such as, but not limited to, hard drives, solid state drives (SSD), flash drives, USB drives, optical drives, and internet-based storage (e.g., “cloud” storage).

In one embodiment, a computer system comprises multiple client devices in communication with one or more server devices through or over a network, although in some cases no server device is used. In various embodiments, the network may comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. The client devices each comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CDROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C#, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RAM typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, personal digital assistants, computer notebooks, hand-held devices, cellular phones, mobile phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A container for use with a personal vaporizing unit, comprising:
an ampoule with a body with a proximal end and distal end, the proximal end comprising an opening; and
a piezoelectric unit attached to said ampoule.

2. The container of claim 1, wherein the piezoelectric unit comprises one or more piezoelectric transducers or atomizers configured to vaporize or atomize a substance with sonic or ultrasonic energy.

3. The container of claim 2, wherein the substance is contained in the body of the ampoule.

4. The container of claim 1, further comprising a cap adapted to cover the opening.

5. The container of claim 4, wherein the cap is removably affixed to the ampoule.

6. The container of claim 5, wherein the cap is permanently affixed to the ampoule.

7. The container of claim 4, wherein the piezoelectric unit is contained in the cap.

8. The container of claim 4, wherein the piezoelectric unit is contained in a holder extending across the opening.

9. The container of claim 1, wherein the piezoelectric unit is in wafer form.

10. The container of claim 2, wherein the ampoule is single-use.

11. The container of claim 2, wherein the ampoule is refillable.

12. The container of claim 1, wherein the piezoelectric unit is embedded in the body of the ampoule.

13. The container of claim 1, wherein the ampoule body comprises an exterior with printing thereon.

14. The container of claim 13, where the printing comprises a bar code or numeric code.
